(19) United States
(12) Patent Application Publication
(10) Pub. No.: US 2026/0083555 A1
(43) Pub. Date: Mar. 26, 2026

(54) OUTER SKIRT FOR PROSTHETIC HEART VALVE

(71) Applicant: Edwards Lifesciences Corporation, Irvine, CA (US)

(72) Inventor: Tamir S. Levi, Zikhron Yaakov (IL)

(73) Assignee: Edwards Lifesciences Corporation, Irvine, CA (US)

(21) Appl. No.: 19/406,802

(22) Filed: Dec. 2, 2025

Related U.S. Application Data
(63) Continuation of application No. PCT/US2024/034679, filed on Jun. 20, 2024.
(60) Provisional application No. 63/522,339, filed on Jun. 21, 2023.

(51) Int. Cl. A61F 2/24

(52) U.S. Cl. CPC A61F 2/2418; A61F 2/2415; A61F 2210/0071; A61F 2220/005; A61F 2220/0075; A61F 2240/001; A61F 2250/0025

(57) ABSTRACT

A prosthetic valve includes a radially expandable and compressible frame, a leaflet assembly comprising a plurality of leaflets coupled to the frame, and an outer skirt positioned around an outer surface of the frame. The outer skirt includes a tissue layer and a non-tissue layer. The tissue layer has a fibrous side and a smooth side. The non-tissue layer is coupled to the smooth side of the tissue layer. The outer skirt is positioned so that the fibrous side of the tissue layer faces away from the frame and the non-tissue layer faces towards the frame.

### CROSS-REFERENCE TO RELATED APPLICATIONS

This application is a continuation of International Patent Application No. PCT/US2024/034679, filed Jun. 20, 2024, which claims the benefit of U.S. Provisional Application No. 63/522,339, filed Jun. 21, 2023, all of which applications are incorporated by reference herein in their entireties.

### FIELD

The present disclosure relates to implantable, radially expandable prosthetic devices, such as prosthetic heart valves, and to methods, assemblies, and apparatuses for delivering, expanding, implanting, and deploying such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve (or simply “prosthetic valve”) can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Despite the recent advancements in percutaneous valve technology, there remains a need for improved transcatheter prosthetic valves.

### SUMMARY

The present disclosure relates to methods and devices for treating valvular diseases. Specifically, the present disclosure is directed to implantable, radially expandable prosthetic devices, such as prosthetic heart valves, and to methods, assemblies, and apparatuses for delivering, expanding, implanting, and deploying such prosthetic devices.

A prosthetic valve can include a radially expandable and compressible frame, and a leaflet assembly comprising a plurality of leaflets coupled to the frame. In addition to these features, a prosthetic valve can further comprise one or more of the components disclosed herein.

In certain examples, the prosthetic valve can include an outer skirt positioned around an outer surface of the frame.

In certain examples, the outer skirt includes a tissue layer and a non-tissue layer.

In certain examples, the tissue layer includes a fibrous side and a smooth side, and the non-tissue layer can be coupled to the smooth side of the tissue layer.

In certain examples, the outer skirt can be positioned so that the fibrous side of the tissue layer faces away from the frame and the non-tissue layer faces towards the frame.

Certain aspects of the disclosure concern a method of making a prosthetic heart valve. The method can include preparing a tissue layer having a fibrous side and a smooth side, forming an outer skirt by attaching a non-tissue layer to the smooth side of the tissue layer, and mounting the outer skirt to an outer surface of an annular frame so that the fibrous side of the tissue layer faces away from the frame and the non-tissue layer faces towards the frame.

Certain aspects of the disclosure concern a method of forming a skirt for a prosthetic valve. The method can include treating a surface of pericardial tissue to increase a surface roughness of the surface.

Certain aspects of the disclosure concern a method of implanting a prosthetic valve. The method includes delivering a prosthetic device in a radially compressed state to a target location, and radially expanding the prosthetic device to a radially expanded state. The prosthetic device can be any one of the prosthetic valves described above. The above method of implanting a prosthetic valve can be performed on a living animal or on a simulation, such as on a cadaver, cadaver heart, anthropomorphic ghost, simulator (for example, with body parts, heart, tissue, etc. being simulated).

In some examples, a prosthetic valve comprises one or more of the components recited in Examples 1-10 and 35-37 described in the section “Additional Examples of the Disclosed Technology”below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1A is a side view of a prosthetic heart valve, according to one example.

FIG. 1B is a side view of a prosthetic heart valve, according to another example.

FIG. 2 is a side view of an exemplary delivery apparatus configured to deliver and implant a radially expandable prosthetic valve at an implantation site.

FIG. 3 is a cross-sectional view of a layer of pericardial tissue.

FIG. 4 is a cross-sectional view of a portion of an example outer skirt that includes the tissue layer of FIG. 3.

FIG. 5 is a flattened view of the outer skirt of FIG. 4.

FIG. 6 is a flattened view of another exemplary outer skirt that includes the tissue layer of FIG. 3.

FIG. 7 shows an exemplary prosthetic heart valve having the outer skirt of FIG. 6.

FIG. 8 illustrates mounting an outer skirt to a prosthetic valve, according to one example.

FIG. 9 illustrates mounting an outer skirt to a prosthetic valve, according to another example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. Further, as used herein, “and/or” means “and” or “or,” as well as “and” and “or.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,” “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

### Overview of Prosthetic Valves

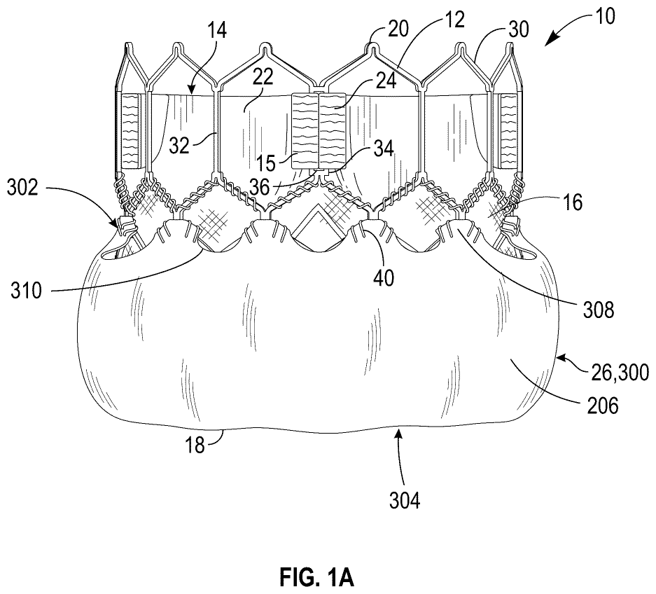

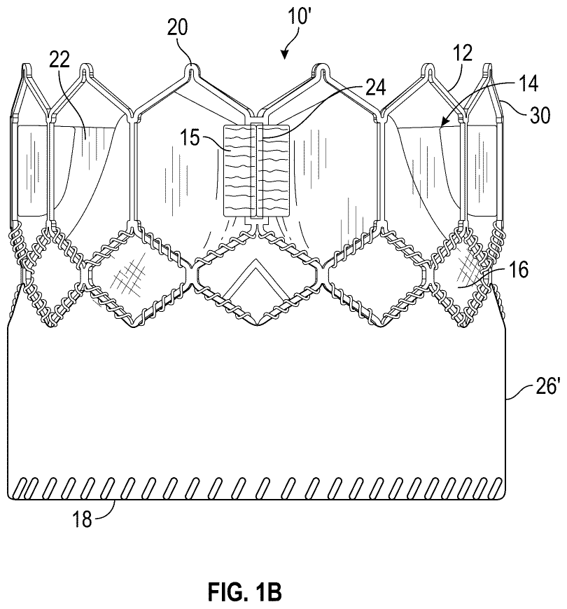

FIG. 1A shows a prosthetic heart valve 10 (or simply, “prosthetic valve”), according to one example. Any of the prosthetic valves disclosed herein can be adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Pat. No. 10,363,130, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 10 can include an annular stent or frame 12, a valvular structure 14, and a perivalvular outer sealing member or outer skirt 26. The prosthetic heart valve 10 (and the frame 12) can have an inflow end 18 and an outflow end 20. The valvular structure 14 can be disposed on an interior of the frame 12 while the outer skirt 26 can be disposed around an outer surface of the frame 12.

The valvular structure 14 can comprise a plurality of leaflets 22 (for example, two, three, or more leaflets), collectively forming a leaflet structure (which can also be referred to as a “leaflet assembly”) that is coupled to the frame 12. The leaflet structure is movable between an open state which permits blood to flow from the inflow end 18 to the outflow end 20 of the frame and a closed state which blocks blood to flow from the outflow end 20 to the inflow end 18 of the frame.

The frame 12 can include multiple rows of angled struts 30. For example, an uppermost row of angled struts can define the outflow end 20, a lowermost row of angled struts can define the inflow end 18, and one or more intermediate rows of angled struts can extend between the uppermost and lowermost rows of angled struts. In some examples, the frame 12 can include a plurality of axial struts bridging two adjacent rows of angled struts. For example, FIG. 1A shows a plurality of axial struts 32 bridging two rows of angled struts that are closest to the outflow end 20.

The leaflets 22 can be secured to one another at their adjacent sides to form commissures 24 of the valvular structure 14. For example, each leaflet 22 can comprise opposing commissure tabs 15 disposed on opposite sides of the leaflet 22 and a cusp edge portion extending between the opposing commissure tabs 15. Selected axial struts, also referred to as window struts 34, can have openings or commissure windows 36 configured to receive commissures 24 of the valvular structure. For example, each commissure window 36 can be configured to receive a pair of commissure tabs 15 of a pair of adjacent leaflets 22 arranged into a commissure 24.

In some examples, the cusp edge portion of the leaflets 22 can have an undulating, curved scalloped shape. In some examples, the cusp edge portion of the leaflet 22 can be secured directly to the frame 12 via one or more fasteners (for example, sutures). In other examples, the cusp edge portion of the leaflets 22 can be secured to an inner skirt 16 which is then secured to the frame 12. Various methods for securing the leaflets 22 to a frame, such as the frame 12, are disclosed in U.S. Pat. No. 9,393,110 and U.S. Provisional Patent Application Nos. 63/278,922, filed Nov. 12, 2021, and 63/300,302, filed Jan. 18, 2022, all of which are incorporated by reference herein. Additional examples of leaflet structure are described in U.S. Provisional Ser. No. 63/448,767 , filed Feb. 28, 2023, which is incorporated by reference herein.

In some examples, the leaflets 22 can be formed of pericardial tissue (for example, bovine pericardial tissue, etc.), biocompatible synthetic materials, or various other suitable natural or synthetic materials as known in the art and described in U.S. Pat. No. 6,730,118, which is incorporated by reference herein.

In some examples, the outer skirt 26 can be an annular skirt. In some instances, the outer skirt 26 can comprise one or more skirt portions that are connected together and/or individually connected to the frame 12. In some examples, the outer skirt 26 can comprise a fabric or polymeric material, such as ePTFE, PTFE, PET, TPU, UHMWPE, PEEK, PE, etc. In some examples, the outer skirt 26 can comprise natural tissue, such as a pericardial tissue (for example, bovine pericardial tissue, porcine pericardial tissue, or pericardial tissue from other sources), as described more fully below.

In some instances, as shown in FIG. 1A and described further below, the outer skirt 26 can have an undulating upper edge portion that extends along and is secured to some angled struts of the frame 12. In other examples, the outer skirt 26 can have a relatively straight upper edge portion. As shown in FIG. 1A, the outer skirt 26 can be sized such that it has slack between the upper and lower edges and can bulge outwardly from the frame 12 when the prosthetic valve is in the radially expanded state. Additional examples of the outer skirts are described in U.S. provisional Ser. No. 63/366,599 filed Jun. 17, 2022, which is incorporated by reference herein.

FIG. 1B shows a prosthetic valve 10′, according to another example. The prosthetic valve 10′ is identical to the prosthetic valve 10 except that the prosthetic valve 10′ includes an outer skirt 26′. Like the outer skirt 26, the outer 26 can be made from natural tissue, such as pericardial tissue. In contrast to the outer skirt 26, the outer skirt 26′ is sized such that it lies against the outer surface of the frame in a snug or tight-fitting manner when the prosthetic valve is in the radially expanded state.

The frame 12 can be radially compressible and expandable between a radially compressed (or collapsed) configuration and a radially expanded configuration (the expanded configuration is shown in FIG. 1). The frame 12 can be made of any of various suitable plastically-expandable materials (for example, stainless steel, etc.) or self-expanding, shape memory materials (for example, nickel titanium alloy (NiTi), such as nitinol). When constructed of a plastically-expandable material, the frame 12 (and thus the valve 10) can be crimped to a radially compressed state on a delivery catheter and then expanded inside a patient by an inflatable balloon or equivalent expansion mechanism. When constructed of a self-expandable material, the frame 12 (and thus the valve 10) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Additional details and examples of frames for prosthetic heart valves can be found in U.S. Pat. No. 9,393,110 and PCT Application No. PCT/US2022/025687, which is incorporated by reference herein.

### Exemplary Delivery Apparatus

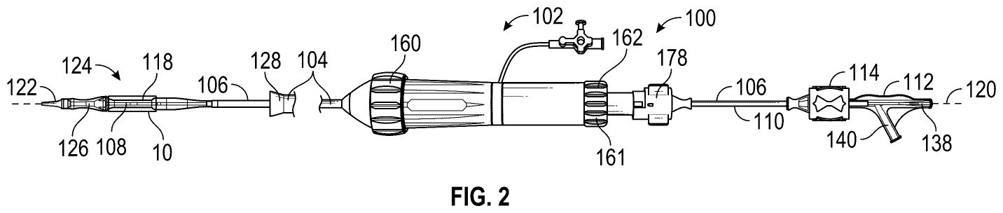

FIG. 2 shows a delivery apparatus 100, according to an example, that can be used to implant an expandable prosthetic valve (for example, the prosthetic valve 10 and/or any of the other prosthetic valves described herein). In some examples, the delivery apparatus 100 can be specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122, or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic valve in a radially compressed state. For example, as shown schematically in FIG. 2, a prosthetic device, such as the prosthetic valve 10, can be mounted around the balloon 118, at the valve mounting portion 124 of the delivery apparatus 100.

The balloon shoulder assembly, including the distal shoulder 126, can be configured to maintain the prosthetic valve 10 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 10 is mounted in the radially compressed state on the valve mounting portion 124 and during delivery of the prosthetic valve to the target implantation site. As such, the distal tip portion 128 can be configured to resist movement of the prosthetic valve 10 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 10.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn can be operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 can be configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

Although the delivery apparatus 100 depicted in FIG. 2 is specifically adapted to deliver a balloon expandable prosthetic valve, it is to be understood that variants of the delivery apparatus 100 can be adapted for delivery of self-expandable prosthetic valves and/or mechanically expandable prosthetic valves, as described in references incorporated above.

### Exemplary Outer Skirts Formed From Natural Tissue

Any of the outer skirts described herein can function as a sealing member for a prosthetic valve by sealing against the tissue of the native valve annulus, helping to reduce paravalvular leakage past the prosthetic valve. Any of the outer skirts described hereinafter can replace the outer skirt 26 of the prosthetic valve 10 or the outer skirt 26′ of the prosthetic valve 10′ described above.

In some examples, an outer skirt for a prosthetic valve can comprise natural tissue. In some examples, the tissue can comprise pericardial tissue harvested from an animal. Although a pericardial tissue is described below as an example used in an outer skirt, it should be understood that other biological tissues or tissue membranes can also be used to form an outer skirt, including dura mater, peritoneum, diaphragm, or tissue from other anatomical sources.

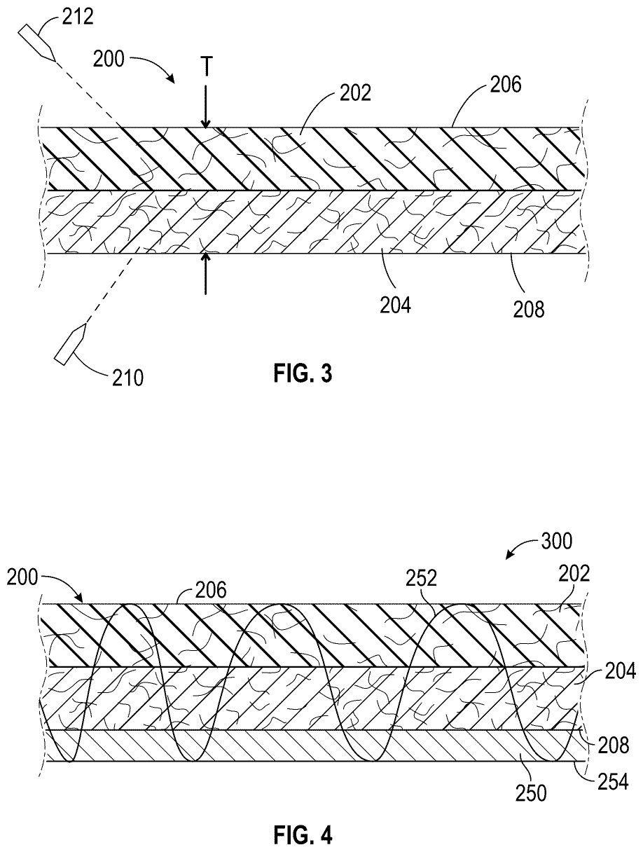

As an example, FIG. 3 shows a cross-sectional view of a patch or section of a pericardial tissue 200 that can be formed into part of an outer skirt. The pericardial tissue 200 can be bovine pericardium, porcine pericardium, equine pericardium, kangaroo pericardium, or pericardium from other sources.

The pericardial tissue 200 has a rough or fibrous layer 202 (also referred to as “fibrous side”) having a relatively rough surface 206 and a smooth layer 204 (also referred to as “smooth side”) having a relatively smooth surface 208 which is on the opposite side of the rough surface 206. The smooth layer 204 (including the smooth surface 208) is smoother and less fibrous than the fibrous layer 202 (including the rough surface 206). In some examples, the pericardial tissue 200 can be formed from a section of the parietal pericardial membrane comprising a fibrous parietal layer (the outermost layer of the pericardium) forming the rough fibrous layer 202 and a serous parietal layer (the outer serous layer) forming the smooth layer 204.

In some examples, the tissue 200 can be harvested and prepared for use in an implant using those techniques and mechanisms known for processing pericardial tissue for heart valve leaflets. A process for preparing pericardial tissue for heart valve leaflets typically includes first obtaining a fresh pericardial sac from a source animal, and then cutting the sac open along predetermined anatomical landmarks to obtain a parietal pericardial membrane. The parietal pericardial membrane can be flattened and typically cleaned of excess fat and other impurities. After trimming obviously unusable areas, a window or patch of tissue can be fixed, typically by immersing in an aldehyde to cross-link the tissue. Rough edges of the tissue window can be removed and the tissue can be bio-sorted to result in a tissue section. The process of bio-sorting involves visually inspecting the window for unusable areas, and trimming the section therefrom. Further details regarding the process for processing pericardial tissue are disclosed in U.S. Pat. Nos. 8,846,390 and 9,358,107, which are incorporated herein by reference in their entirety.

In certain examples, following initial processing of the pericardial tissue as described in the preceding paragraph, the overall thickness of the pericardial tissue can be reduced by removing a portion of the pericardial tissue 200, such as by using a laser 210 and/or laser 212 in a laser milling process, until the pericardial tissue has a desired thickness T. In some embodiments, the final thickness T after milling is between 50 μm and 100 μm, inclusive. Alternatively, the pericardial tissue 200 can be milled or otherwise formed to any other thickness T. After the tissue is processed, the pericardial tissue 200 has a smooth layer 204, a rough fibrous layer 202, a thickness T, and it can be formed into an outer skirt as discussed further below.

In certain examples, the laser 210 can be a non-thermal laser such as a femtosecond laser. In such circumstances, the femtosecond laser can be applied to the smooth layer 204 of the pericardial tissue 200. Relative movement between the femtosecond laser and the pericardial tissue 200 can be actuated to ablate material from the smooth layer 204. As a result, the overall thickness of the pericardial tissue 200 can be reduced by removing a partial thickness of the smooth layer 204. The laser milled smooth layer 204 can still result in a smooth surface 208 while the fibrous layer 202 on the opposite side retains its rough surface 206.

In one specific example, the smooth layer 204 can be completely removed by the laser 210. In such circumstances, the pericardial tissue 200 is left with only the fibrous layer 202, which has rough surfaces on both sides (that is, the smooth surface 208 is replaced with another rough surface similar to 206).

The femtosecond laser typically uses laser pulses of ultra-short durations (for example, one the order of femtoseconds) such that the laser energy is deposited into the target material so quickly that there is not enough time for significant heat to accumulate. Instead, the laser energy is primarily used to excite electrons in the target material, leading to non-thermal processes such as photochemical reactions and ionization.

In some examples, the laser 212 can be a thermal laser such as a gas laser, a solid-state laser, a semiconductor laser, a fiber laser, etc., configured to heat the target tissue. In one specific example, the laser 212 can be a carbon dioxide (CO2) laser. In some examples, a femtosecond laser may be modified to induce thermal damage. In contrast to the non-thermal laser, the thermal laser can generate a sufficient heat on the target tissue which can change surface properties of the target tissue to encourage a biological response when the prosthetic valve is implanted within a native heart valve, for example, causing calcium deposition and/or pannus formation over time to promote sealing of the prosthetic valve against the surrounding tissue. In such circumstances, the thermal laser can be applied to the fibrous layer 202 of the pericardial tissue 200. Relative movement between the thermal laser and the pericardial tissue 200 can be actuated to ablate material from the fibrous layer 202. As a result, the overall thickness of the pericardial tissue 200 can be reduced by removing a partial thickness of the fibrous layer 202. The laser milled fibrous layer 202 can still have a rough surface 206 (for example, due to the surface irregularities formed by the thermal laser) while the smooth layer 204 on the opposite side retains its smooth surface 208.

In some examples, the overall thickness of the pericardial tissue 200 can be reduced by applying a non-thermal laser (for example, a femtosecond laser) on the smooth layer 204 and independently applying a thermal laser (for example, a CO2 laser) on the fibrous layer 202, as described above. In some examples, the desired overall thickness T of the tissue 200 can be achieved primarily via application of the laser 210 to the smooth layer 204, while the laser 212 can be used to create surface irregularities on the rough surface 206 without reducing the thickness of the layer 202 or while substantially maintaining the thickness of the layer 202. In some examples, the overall thickness T of the pericardial tissue 200 can be achieved with the application of the laser 212 to the layer 202.

In addition to, and/or in lieu of, laser tissue removal described above, various mechanical devices for skiving or shaving tissue such as razor or planing devices may be used to remove some of the pericardial tissue. For instance, a device having a flat platen over which a planing razor or blade translates may be substituted for the laser ablation described above. Other physical configurations for creating relative tissue/razor movement can be used, for instance using a lathe-like razor to smooth the smooth surface 208. Each of these devices may be automatically or computer-controlled using an optical surface measuring component to control the depth of cut. Abrasive tissue removal (e.g., sanding or rasping) can also be used. The grit of the tool can be relatively fine for removing tissue material from the smooth layer 204 or relatively coarse for removing tissue material from the fibrous layer 202. In other examples, a dermatome can be used for skiving or shaving of a portion of the smooth layer 204. Further details regarding the use of a dermatome for removing portions of tissue from pericardial tissue are disclosed in U.S. Pat. No. 8,846,390.

In some examples, in lieu of or in addition to the use of a thermal laser 212, the rough surface 206 of the fibrous layer 202 can be further frayed or roughened to increase its surface area. For example, the roughness of the rough surface 206 can be increased by fraying the fibrous layer 202 via chemical treatment. As another example, the roughness of the rough surface 206 can be increased by mechanically altering the fibrous layer 202 by scraping the surface 206 with a tool, such as a brush or a combing device, or by sanding or rasping the layer 202.

In some examples, the pericardial tissue 200, by itself, can form an outer skirt (for example, the outer skirt 26 or the outer skirt 26′) for a prosthetic valve (for example, the prosthetic valve 10 or 10′). In such examples, when mounting the outer skirt to an outer surface of a frame (for example, the frame 12) of the prosthetic valve, the outer skirt is positioned so that the fibrous layer 202 (including the rough surface 206) of the pericardial tissue 200 faces away from the frame and the smooth layer 204 (including the smooth surface 208) of the pericardial tissue 200 faces towards the frame. As such, when the prosthetic heart valve is implanted in a patient, the rough surface 206 of the pericardial tissue 200 faces the native tissue of the patient. The rough surface 206 facing or being in contact with the native tissue can help disturb blood flow between the outer skirt and the native anatomy of the patient, which can enhance tissue ingrowth and proliferation and help seal any gaps between the prosthetic heart valve and the native anatomy to reduce and/or eliminate perivalvular leakage. In contrast, the smooth surface 208, facing inwardly, is configured to be more resistant to thrombosis. In addition, if the outer skirt protrudes inwardly through the frame during cycling or when the prosthetic heart valve is crimped in a radially collapsed configuration, any contact between a valvular structure (for example, the valvular structure 14) of the prosthetic valve and the outer skirt will be with the smooth surface 208 of the pericardial tissue 200, which can be less abrasive than outer skirts made of PET or some other fabrics. Thus, the outer skirt made from pericardial tissue 200 can help protect the leaflets of the valvular structure. Additional examples of forming an outer skirt using a pericardial tissue are described in U.S. Pat. No. 10,973,628, which is incorporated herein by reference.

In some examples, any of the surface treatments described above to fray the tissue and/or produce surface irregularities (treatment by application of a thermal layer, chemical treatment, and/or mechanical treatment) can be applied to the smooth surface 208 of the layer 204 of the pericardial tissue, or to both the surface 208 and the surface 206.

In some examples, the outer skirt for a prosthetic valve can comprise the pericardial tissue 200 mounted on the outer surface of the frame (for example, frame 12) with the smooth surface 208 facing outwardly away from the frame for contacting native tissue and the rough surface 206 facing inwardly toward the frame. In such examples, the surface 208 can be untreated or it can be treated with one or more of the surface treatments described above.

In some examples, the pericardial tissue 200 can be used in combination with other material layers to form an outer skirt (for example, the outer skirt 26 or the outer skirt 26′) for a prosthetic valve (for example, the prosthetic valve 10 or 10′). For example, FIG. 4 shows an outer skirt 300 that includes a non-tissue layer 250 disposed against the pericardial tissue 200.

In the example depicted in FIG. 4, the non-tissue layer 250 can comprise a smooth polymeric layer made from, for example, polytetrafluoroethylene (PTFE), expanded PTFE (ePTFE), thermoplastic polyurethane (TPU), silicone, and the like. In some examples, the layer 250 comprises a non-woven or non-textile construction, such as may be formed by extruding or molding the polymeric material into a layer of such material. In other examples, the layer 250 can comprise a textile, such as a woven, knitted, or braided layer formed from any of various synthetic (e.g., polymeric) or natural yarns or fibers. When the layer 250 comprises a textile, the layer 250 desirably comprises a weave that provides a relatively smooth surface on one or both sides of the layer, such as a satin or velvet weave. In some examples, the layer 250 has a thickness ranging between 10 μm and 50 μm, inclusive.

The layer 250 can be attached to the smooth layer 204 of the pericardial tissue 200. In one example, as shown in FIG. 4, the layer 250 can be fixedly attached to the pericardial tissue 200 via one or more sutures 252 forming stitches extending through the layer 250 and the tissue 200. In another example, the layer 250 can be glued or adhered to the smooth layer 204 of the pericardial tissue 200 via an adhesive. In still another example, both sutures 252 and an adhesive can be used to affix the layer 250 to the pericardial tissue 200. Thus, the outer skirt 300 retains the rough surface 206 of the fibrous layer 202 on one side and a smooth surface 254 formed by the layer 250 on the opposite side. In some examples, the smooth surface 254 is smoother and therefore has a smaller surface area than the rough surface 206.

In one specific example, the pericardial tissue 200 only has the fibrous layer 202 (for example, the smooth layer 204 is completely removed by laser milling). In such circumstances, the layer 250 can be directly attached to the fibrous layer 202 (for example, via sutures and/or an adhesive). Again, the resulting outer skirt 300 retains the rough surface 206 of the fibrous layer 202 on one side and the smooth surface 254 formed by the layer 250 on the opposite side.

Sometimes, it may become medically necessary to explant a previously implanted prosthetic valve having the outer skirt 300. Explantation of the prosthetic valve can be challenging if neointimal tissue has been formed between the fibrous layer 202 of the pericardial tissue 200 and the surrounding native anatomy over time. In some circumstances, the layer 250 can be configured to be detachable from the pericardial tissue 200 to facilitate explantation of prosthetic valve, if necessary. Specifically, the layer 250 can be detached from the pericardial tissue 200 and removed from the patient's body, together with other components of the prosthetic valve that are covered by the layer 250 (for example, the frame, the leaflet structure and inner skirt within the frame). For example, the sutures 252 used to attach the layer 250 to the pericardial tissue 200 can be bioabsorbable such that the sutures 252 can dissolve and be absorbed within the body after a certain period of time. As another example, the sutures 252 used to attach the layer 250 to the pericardial tissue 200 can be removed after implantation of the prosthetic valve, as taught by the PCT Patent Application No. PCT/US2022/049661, which is incorporated herein by reference. Because the surface of the layer 250 in contact with the pericardial tissue is relatively smooth and can prevent or minimize the formation of tissue at the interface between the layer 250 and the pericardial tissue, the layer 250 (along with the frame and other components of the prosthetic valve) can be easily separated from the pericardial tissue 200 and removed from the patient once the suture 252 dissolves in the body or is otherwise removed. In some such examples, the outer skirt 300 can be initially attached to the frame of the prosthetic with sutures that form stitches that extend through the layer 250 and around struts of the frame (and/or through an inner skirt) but do not extend through the pericardial tissue 200.

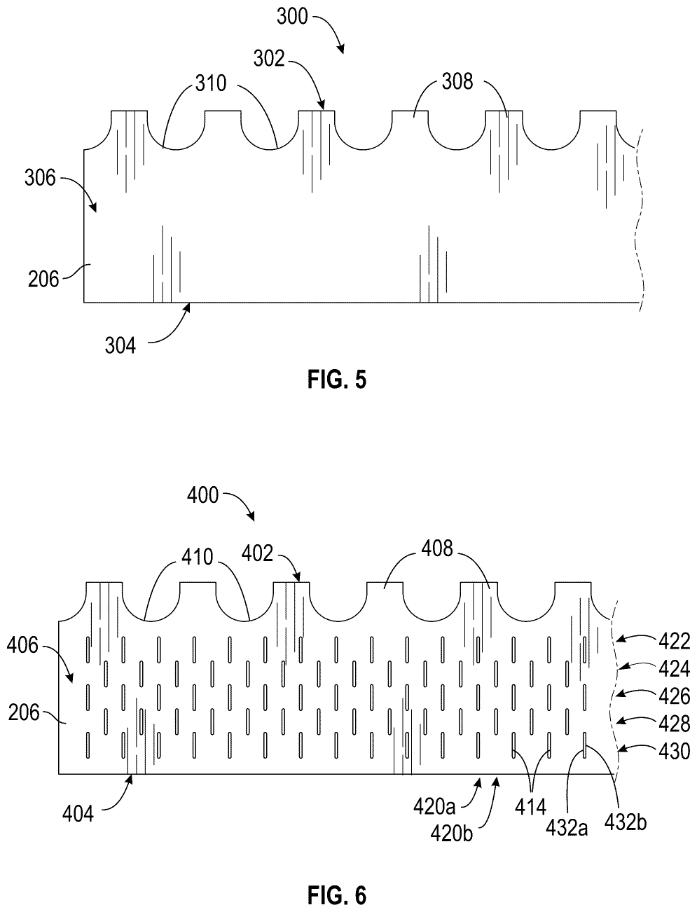

FIG. 5 is a flattened view of the outer skirt 300 prior to its attachment to the frame of a prosthetic heart valve (the figure shows the rough surface 206 while the smooth surface 254 is hidden from the view). The outer skirt 300 can be one example of the outer skirt 26 depicted in FIG. 1A. In other examples, an outer skirt comprising a tissue layer 200 and a non-tissue layer 250 can have the same overall shape and size of the skirt 26′ of FIG. 1B.

Referring to FIG. 5, the outer skirt 300 can have a first end portion 302 (i.e., the upper end portion as depicted in FIG. 5; also referred to as the “outflow end portion” in the illustrated example), a second end portion 304 (i.e., the lower end portion as depicted in FIG. 5; also referred to as the “inflow end portion” in the illustrated example), and an intermediate portion 306 disposed between the first and second end portions 302, 304. In the depicted example, the first end portion 302 of the outer skirt 300 can include a plurality of alternating projections 308 and notches 310, or castellations. In other examples, first end portion 302 can be formed without any projections 308 or notches 310 and instead can be substantially straight.

As depicted in FIG. 1A, the outer skirt 300 can be attached to the prosthetic heart valve 10. The projections 308 of the first end portion 302 can be attached to the inner skirt 16 and/or the frame 12 of the prosthetic heart valve 10 using sutures 40 and/or an adhesive. The second end portion 304 can be attached to the inner skirt 16 and/or the frame 12 of the prosthetic heart valve 10 using sutures, adhesive, or any other suitable attachment means.

In the illustrated example of FIG. 1A, the outer skirt 300 can be secured to the frame 12 with the rough surface 206 of the pericardial tissue 200 facing away from the frame 12 and the smooth surface 254 of the layer 250 facing the frame 12. As such, when the prosthetic valve 10 having the outer skirt 300 is implanted in a patient, the rough surface 206 of the pericardial tissue 200 faces the native tissue of the patient. The rough surface 206 facing or being in contact with the native tissue can disturb the blood flow between the outer skirt 300 and the native anatomy of the patient. Such blood disturbance can induce a thicker boundary layer which can result in local blood stasis and enhance tissue ingrowth and proliferation. Further, the rough surface 206 exposes a larger surface area of the material surrounding the prosthetic valve 10 to interact with the blood, which also encourage tissue ingrowth. The tissue ingrowth over the rough surface 206 in turn can help seal any gaps between the prosthetic heart valve 10 and the native anatomy to reduce and/or eliminate perivalvular leakage. In contrast, the smooth surface 254 of the layer 250, facing inwardly, is configured to be more resistant to thrombosis.

The attachment of the layer 250 to the pericardial tissue 200 can also protect the integrity of the outer skirt 300 and/or the valvular structure 14. For example, during cycling or when the prosthetic heart valve 10 is crimped in a radially collapsed configuration, the layer 250 can function as a barrier to prevent portions of the pericardial tissue 200 from protruding inwardly through opening cells of the frame 12, thereby reducing the risk of abrasion or pinching of the pericardial tissue 200 by the struts of the frame 12. Additionally, the smooth layer 250 can prevent the pericardial tissue 200 (including the fibrous layer 202) from rubbing against the leaflets 22 (especially in configurations where the inner skirt 16 is lower than the outer skirt 300), thereby reducing the risk of abrasion to the leaflets 22.

FIG. 6 is a flattened view of another outer skirt 400 prior to its attachment to the frame of a prosthetic valve. Similar to the outer skirt 300, the outer skirt 400 comprises a pericardial tissue 200 and a non-tissue layer 250. In the example depicted in FIG. 6, the rough surface 206 of the fibrous layer 202 is shown while the smooth surface 254 of the layer 250 is hidden from the view.

Similar to the outer skirt 300, the outer skirt 400 can include a first end portion 402 (i.e., the upper end portion as depicted in FIG. 6), a second end portion 404 (i.e., the lower end portion as depicted in FIG. 6), and an intermediate portion 406 disposed between the first and second end portions 402, 404. In the depicted example, the first end portion 402 of the outer skirt 400 can include a plurality of alternating projections 408 and notches 410, or castellations. In other examples, the first end portion 402 can be formed without any projections 408 or notches 410 and instead can be substantially straight.

As shown in FIG. 6, the intermediate portion 406 can include a plurality of slits or openings 414. The slits 414 can be cut or otherwise formed in a longitudinal direction (i.e., an axial direction when the outer skirt 400 is attached to the frame of a prosthetic heart valve). The slits 414 can be laser cut or formed by any other means. For example, the slits 414 can be cut by using a non-thermal laser (similar to the laser 210 depicted in FIG. 3), such as a femtosecond laser). As another example, the slits 414 can be cut by using a thermal laser (similar to the laser 212 depicted in FIG. 3), such as a CO2 laser. In certain examples, the slits 414 can extend through a thickness of the pericardial tissue 200 but not the layer 250.

In the example depicted in FIG. 6, the slits 414 are elongated axially and can be arranged in five rows 422, 424, 426, 428, and 430. In other examples, the slits 414 can be arranged in more or less than five rows. In the depicted example, the rows 422, 426, and 430 of slits 414 are circumferentially aligned with each other and are offset from rows 424 and 428 of slits 414, which are circumferentially aligned with each other. In other examples, alignments between the rows can be varied.

In some examples, each slit 414 includes first and second opposing longitudinal sides 432a, 432b, respectively, that are spaced apart from each other to define a permanent open gap therebetween. In other examples, the longitudinal sides 432a, 432b of a slit 414 are in contact with each other (and do not define a permanent open gap therebetween) in the absence of hemodynamic forces, but can move away from each other under hemodynamic forces to allow blood to flow through the pericardial tissue 200 via the slits 414. In certain examples, the layer 250 is non-porous such that blood flowing through the pericardial tissue 200 via the slits 414 does not penetrate the layer 250.

In the example depicted in FIG. 6, the slits 414 are arranged in alternating axially extending columns 420a and 420b. The columns 420a can each have three slits and the columns 420b can each have two slits. In other examples, the slits 414 can be arranged on the outer skirt 400 in any pattern including any number of rows and/or columns containing any number of slits or any other pattern not having a particular number of rows and/or columns. Alternatively, the slits 414 can be arranged on the outer skirt 400 in a way that does not have a particular pattern. In some examples, the slits or openings 414 can have any of various other shapes, such as circular, square, rectangular, triangular, or various combinations thereof. In some examples, the slits or openings 414 can be elongated circumferentially or at any other angle with respect to the orientation of the outer skirt 400.

FIG. 7 shows the outer skirt 400 attached to the prosthetic heart valve 10, similar to the example described above with reference to FIG. 1A. As noted above, when the prosthetic valve 10 is implanted in a patient, the rough surface 206 of the pericardial tissue 200 can help reducing and/or eliminating perivalvular leakage. Additionally, blood can flow through the slits 414, which can slow the flow of antegrade blood and further enhance blood clotting and tissue ingrowth, which can further help to prevent perivalvular leakage. Furthermore, the longitudinal or axial direction of the slits 414 can help reduce stretching or deformation of the outer skirt 400 during passage through a sheath as may be caused by friction between the outer skirt and an inner surface of the sheath. Again, it should be noted that while outer skirt 400 is illustrated as being attached somewhat loosely, that is, with some slack in the intermediate portion 406 of the outer skirt 400, it can also be attached so as to fit more snugly against the outer surface of the frame 12, such as the skirt 26′ in FIG. 1B.

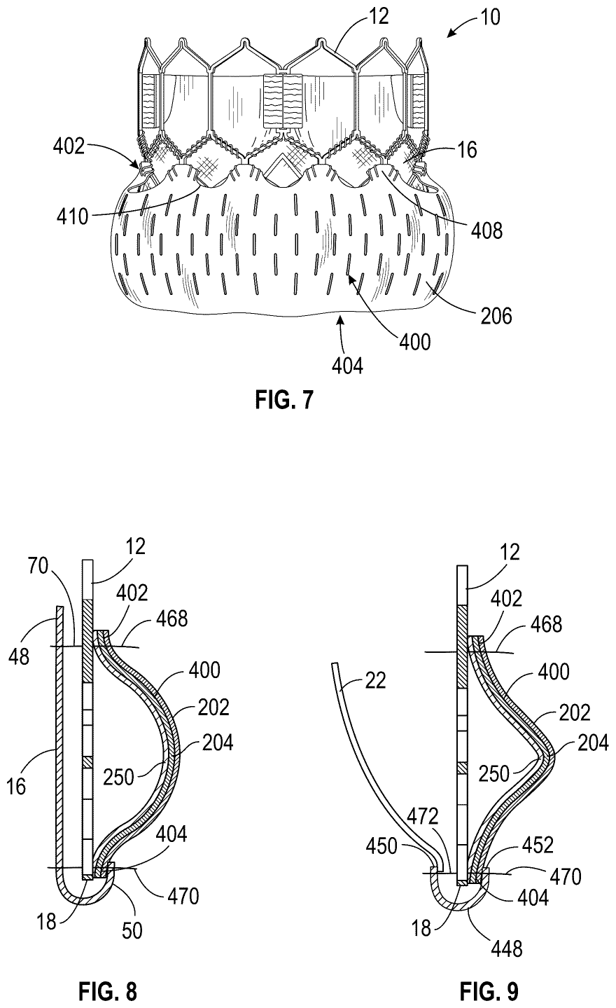

FIGS. 8-9 show various ways of mounting an outer skirt (e.g., outer skirt 300 or outer skirt 400) to the frame 12 of a prosthetic valve 10. For purposes of illustration, reference number 400 is used to designate the outer skirt in FIGS. 8-9, although it should be understood that the other outer skirts (for example, 26, 300) disclosed herein can be mounted to the frame 12 in the same manner.

Referring to FIG. 8, the inner skirt 16 has an upper edge portion 48 and a lower edge portion 50. The upper edge portion 48 of the inner skirt 16 can be secured to the inside of the frame 12, for example, via sutures 70. Alternatively, the upper edge portion 48 of the inner skirt 16 can be secured to the inside of frame 12 via adhesive and/or ultrasonic welding in addition to or in lieu of the sutures 70. The upper edge portion 402 of the outer skirt 400 can be secured to the frame 12 with sutures 468. The upper edge portions 48 and 402 are shown loosely attached to the frame in FIG. 8 for purposes of illustration, but typically can be tightly secured to the frame struts as depicted in FIGS. 1A-1B.

The lower edge portion 50 of the inner skirt 16 can be wrapped around the inflow end 18 of the frame 12 and around the lower edge portion 404 of the outer skirt 400. The lower edge portion 404 of the outer skirt 400 and the wrapped lower edge portion 50 of the inner skirt 16 can be secured together and/or secured to the frame 12, such as with sutures 470 and/or an adhesive. Wrapping the lower edge portion 50 of the inner skirt 16 around the lower edge portion 404 of the outer skirt 400 can reinforce the lower edge portion 404 and the sutures 470 along the lower edge portion 404. The lower edge portions 50 and 404 are shown loosely attached to the frame in FIG. 8 for purposes of illustration, but typically can be tightly secured to the frame struts with the sutures 470.

FIG. 9 shows another way of mounting the outer skirt 400 to the frame 12. The upper edge portion 402 of the outer skirt 400 can be mounted to the frame 12 with sutures 468 as described above. A reinforcing strip 448 having a first edge portion 450 and a second edge portion 452 can be wrapped around the inflow end 18 of the frame 12. The reinforcing strip 448 can be made of fabric material (e.g., PET) or natural tissue (e.g., pericardial tissue). In some examples, the reinforcing strip 448 can be used to secure the cusp portion of each leaflet 22 to the frame 12.

Although not shown in FIG. 9, in some examples, an inner skirt 16 also can be mounted inside of the frame 12. In some examples, the reinforcing strip 448 can be part of the inner skirt 16 that varies in height around the circumference of the inner skirt with a maximum height at the commissures of the leaflets (such as illustrated in FIG. 8) and a minimum height at a location equidistant between two commissures (such as illustrated in FIG. 9). Further details of a reinforcing strip that is used to attach the cusp portions of the leaflets to a frame and details of an inner skirt that has a maximum height at the commissures of the leaflets and a minimum height between the commissures are provided in U.S. Publication No. 2021/0259832, which is incorporated herein by reference in its entirety.

The first edge portion 450 of the reinforcing strip 448 can be positioned inside of the frame 12 while the second edge portion 452 can be positioned outside the frame 12. The first and second edge portions 450, 452 can be attached to each other and/or to the frame 12, using sutures 470 and/or an adhesive. The edge portions 450, 452 are shown loosely attached to the frame in FIGS. 8-9 for purposes of illustration, but typically can be tightly secured to the frame.

The second edge portion 452 of the reinforcing strip 448 can be wrapped around the lower edge portion 404 of the outer skirt 400 such that the lower edge portion 404 is between the frame 12 and the reinforcing strip 448. The lower edge portion 404 of the outer skirt 400 can be secured to the frame 12 and the second edge portion 452 of the reinforcing strip 448 with the sutures 470 and/or an adhesive. As depicted in FIG. 9, the lower cusp portion of each leaflet 22 can be secured between the frame 12 and the first edge portion 450 of the reinforcing strip 448 with sutures (e.g., with sutures 472) and/or an adhesive.

In FIGS. 8-9, the non-tissue layer 250 wraps around the outer surface of the frame 12, and can function as a barrier to prevent portions of the pericardial tissue 200 from protruding inwardly through opening cells of the frame during cycling or when the prosthetic heart valve 10 is radially crimped. As a result, the risk of abrasion or pinching of the pericardial tissue 200 (for example, by the struts of the frame 12) and/or the leaflets 22 (for example, by the outer skirt 400) can be reduced, as described above.

### Exemplary Delivery Techniques

For implanting a prosthetic valve within the native aortic valve via a transfemoral delivery approach, the prosthetic valve can be mounted in a radially compressed state along the distal end portion of a delivery apparatus. The prosthetic valve and the distal end portion of the delivery apparatus can be inserted into a femoral artery and then advanced into and through the descending aorta, around the aortic arch, and through the ascending aorta. The prosthetic valve can be positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) can be introduced into the left ventricle through a surgical opening in the chest and the apex of the heart and the prosthetic valve can be positioned within the native aortic valve. Alternatively, in a transaortic procedure, a prosthetic valve (on the distal end portion of the delivery apparatus) can be introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, into the right atrium, across the atrial septum (for example, through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) can be introduced into the left ventricle through a surgical opening in the chest and the apex of the heart and the prosthetic valve can be positioned within the native mitral valve.

For implanting a prosthetic valve within the native tricuspid valve, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, and into the right atrium, and the prosthetic valve can be positioned within the native tricuspid valve. A similar approach can be used for implanting the prosthetic valve within the native pulmonary valve or the pulmonary artery, except that the prosthetic valve can be advanced through the native tricuspid valve into the right ventricle and toward the pulmonary valve/pulmonary artery.

Another delivery approach is a trans-atrial approach whereby a prosthetic valve (on the distal end portion of the delivery apparatus) can be inserted through an incision in the chest and an incision made through an atrial wall (of the right or left atrium) for accessing any of the native heart valves. Atrial delivery can also be made intravascularly, such as from a pulmonary vein. Still another delivery approach is a trans-ventricular approach whereby a prosthetic valve (on the distal end portion of the delivery apparatus) can be inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Exemplary Materials and Expansion Mechanisms for Prosthetic Valves

For any of the prosthetic valves described herein, the frame can be made of any of various suitable plastically-expandable materials. When constructed of a plastically-expandable material, the frame (and thus the prosthetic valve) can be crimped to a radially collapsed configuration on a delivery catheter or apparatus and then expanded inside a patient by an inflatable balloon or equivalent expansion mechanism. In certain examples, during delivery to the implantation site, the prosthetic valve can be placed inside of a delivery capsule or sheath to protect against the prosthetic valve contacting the patient's vasculature, such as when the prosthetic valve is advanced through a femoral artery. The capsule can also retain the prosthetic valve in a radially compressed state having a slightly smaller diameter and crimp profile than may be otherwise possible without a capsule by preventing any recoil (expansion) of the frame once it is crimped onto the delivery apparatus.

Suitable plastically-expandable materials that can be used to form the frames disclosed herein (for example, the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details on balloon expandable prosthetic valves can be found in U.S. U.S. Pat. No. 9,393,110, and U.S. Provisional Application Nos. 63/178,416, filed Apr. 22, 2021, 63/194,830, filed May 28, 2021, and 63/279,096, filed Nov. 13, 2021, all of which are incorporated by reference herein.

Any of the prosthetic valves described herein can be self-expandable. For example, the frame of the prosthetic valve can comprise a shape-memory material (for example, Nitinol). When the prosthetic valve is self-expandable, the frame (and thus the prosthetic valve) can be crimped to a radially compressed configuration and restrained in the compressed configuration by insertion into a sheath or equivalent mechanism of a delivery catheter. Once inside the body at the desired implantation site, the prosthetic valve can be deployed or released from the delivery sheath, which allows the prosthetic valve to expand to its functional size. In some examples, the frame (and therefore the prosthetic valve) can partially self-expand from the radially compressed configuration to a partially radially expanded configuration. The frame (and therefore the prosthetic valve) can be further radially expanded from the partially expanded configuration to a further radially expanded configuration via one or more actuation assemblies (for example, an inflatable balloon and/or one or more mechanical actuators) of the delivery apparatus.

Additional details regarding exemplary self-expandable prosthetic valves and the related delivery apparatus/catheters/systems are described in U.S. Pat. Nos. 8,652,202, 9,155,619, and 9,867,700, all of which are incorporated herein by reference.

Additionally, and/or alternatively, any of the prosthetic valves described herein can be mechanically expandable. For example, the struts of the frame can be pivotably coupled to one another at one or more pivot joints along the length of each strut. An axial force applied to the frame (for example, pressing the inflow end and the outflow end of the frame toward each other or pulling the inflow end and the outflow end of the frame away from each other) can cause the prosthetic valve to radially expand or compress. The axial force can be generated by actuating one or more mechanical actuators of the delivery apparatus that are operatively coupled to the frame.

Additional details regarding exemplary mechanically-expandable prosthetic valves and the related delivery apparatus/catheters/systems are described in U.S. Patent Application Publication Nos. 2018/0153689, 2018/0311039, 2019/0060057, and PCT Patent Application Publication No. WO/2021/188476, all of which are incorporated by reference herein.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising: a radially expandable and compressible frame; a leaflet assembly comprising a plurality of leaflets coupled to the frame; and an outer skirt positioned around an outer surface of the frame, wherein the outer skirt comprises a tissue layer and a non-tissue layer, wherein the tissue layer comprises a fibrous side and a smooth side, wherein the non-tissue layer is coupled to the smooth side of the tissue layer, and wherein the outer skirt is positioned so that the fibrous side of the tissue layer faces away from the frame and the non-tissue layer faces towards the frame.

Example 2. The prosthetic valve of any example herein, particularly example 1, wherein the tissue layer comprises pericardial tissue.

Example 3. The prosthetic valve of any example herein, particularly any one of examples 1-2, wherein the non-tissue layer is fixedly attached to the tissue layer via one or more sutures.

Example 4. The prosthetic valve of any example herein, particularly any one of examples 1-3, wherein the non-tissue layer is attached to the smooth side of the tissue layer via an adhesive.

Example 5. The prosthetic valve of any example herein, particularly any one of examples 1-4, wherein the tissue layer has a thickness between 50 μm and 100 μm, inclusive.

Example 6. The prosthetic valve of any example herein, particularly example 5, wherein the tissue layer is milled to the thickness by applying a femtosecond laser to the smooth side of the tissue layer.

Example 7. The prosthetic valve of any example herein, particularly any one of examples 5-6, wherein the tissue layer is milled to the thickness by applying a thermal laser to the fibrous side of the tissue layer.

Example 8. The prosthetic valve of any example herein, particularly any one of examples 1-7, wherein the non-tissue layer comprises any one of polytetrafluoroethylene (PTFE), thermoplastic polyurethane (TPU), and silicone.

Example 9. The prosthetic valve of any example herein, particularly any one of examples 1-8, wherein the tissue layer comprises a plurality of slits through which blood can move from the fibrous side to the smooth side such as to promote tissue ingrowth within the plurality of slits.

Example 10. The prosthetic valve of any example herein, particularly example 9, wherein the plurality of slits are elongated in an axial direction.

Example 11. A method of making a prosthetic heart valve, comprising: preparing a tissue layer comprising a fibrous side and a smooth side; forming an outer skirt by attaching a non-tissue layer to the smooth side of the tissue layer; and mounting the outer skirt to an outer surface of an annular frame so that the fibrous side of the tissue layer faces away from the frame and the non-tissue layer faces towards the frame.

Example 12. The method of any example herein, particularly example 11, further comprising attaching a leaflet structure to the frame.

Example 13. The method of any example herein, particularly any one of examples 11-12, wherein the tissue layer comprises pericardial tissue and the act of preparing the tissue layer comprises cutting and fixing the pericardial tissue.

Example 14. The method of any example herein, particularly any one of examples 11-13, wherein the act of forming the outer skirt comprises stitching the non-tissue layer to the tissue layer with one or more sutures.

Example 15. The method of any example herein, particularly any one of examples 11-14, wherein the act of forming the outer skirt comprises gluing the non-tissue layer to the smooth side of the tissue layer.

Example 16. The method of any example herein, particularly any one of examples 11-15, wherein the act of preparing the tissue layer comprises laser milling the tissue layer until the tissue layer has a thickness between 50 μm and 100 μm, inclusive.

Example 17. The method of any example herein, particularly example 16, wherein the act of laser milling comprises applying a femtosecond laser to the smooth side of the tissue layer.

Example 18. The method of any example herein, particularly any one of examples 16-17, wherein the act of laser milling comprises applying a thermal laser to the fibrous side of the tissue layer.

Example 19. The method of any example herein, particularly example 18, wherein the thermal laser comprises a carbon dioxide laser.

Example 20. The method of any example herein, particularly any one of examples 11-19, wherein the act of preparing the tissue layer comprises fraying the fibrous side of the tissue layer via chemical modification.

Example 21. The method of any example herein, particularly any one of examples 11-20, wherein the act of preparing the tissue layer comprises fraying the fibrous side of the tissue layer by mechanically altering the fibrous side.

Example 22. The method of any example herein, particularly any one of examples 11-21, wherein the act of preparing the tissue layer comprises forming a plurality of slits on the tissue layer through which blood can move from the fibrous side to the smooth side.

Example 23. The method of any example herein, particularly example 22, wherein the plurality of slits are elongated in an axial direction when the outer skirt is mounted to the outer surface of the annular frame.

Example 24. The method of any example herein, particularly any one of examples 22-23, wherein the act of forming the plurality of slits comprises applying a femtosecond laser to selected portions of the tissue layer.

Example 25. A method of forming a skirt for a prosthetic valve, the method comprising treating a surface of pericardial tissue to increase a surface roughness of the surface.

Example 26. The method of any example herein, particularly example 25, wherein the act of treating comprises applying a laser to the surface to create surface irregularities.

Example 27. The method of any example herein, particularly example 26, wherein the laser comprises a thermal laser.

Example 28. The method of any example herein, particularly example 25, wherein the act of treating comprises chemically treating the surface.

Example 29. The method of any example herein, particularly example 25, wherein the act of treating comprises mechanically altering the surface.

Example 30. The method of any example herein, particularly example 29, wherein the mechanically altering the surface comprises scraping the surface with a tool.

Example 31. The method of any example herein, particularly any one of examples 25-30, wherein the surface is the surface of a fibrous parietal layer of the pericardial tissue.

Example 32. The method of any example herein, particularly any one of examples 25-30, wherein the surface is the surface of a serous parietal layer of the pericardial tissue.

Example 33. The method of any example herein, particularly any one of examples 25-32, further comprising attaching a non-tissue layer to another surface of the pericardial tissue opposite the treated surface.

Example 34. The method of any example herein, particularly example 35, wherein the non-tissue layer comprises any one of polytetrafluoroethylene (PTFE), thermoplastic polyurethane (TPU), and silicone.

Example 35. A prosthetic valve, comprising: a radially expandable and compressible frame; a leaflet assembly comprising a plurality of leaflets coupled to the frame; and an outer skirt positioned around an outer surface of the frame, wherein the outer skirt comprises a tissue layer and a non-tissue layer.

Example 36. The prosthetic valve of any example herein, particularly example 35, wherein the tissue layer comprises a fibrous side and a smooth side, wherein the non-tissue layer is coupled to the smooth side of the tissue layer, wherein the outer skirt is positioned so that the fibrous side of the tissue layer faces away from the frame and the non-tissue layer faces towards the frame.

Example 37. The prosthetic valve of any example herein, particularly any one of examples 35-36, wherein the non-tissue layer covers at least a majority of the tissue layer.

Example 38. A method of making a prosthetic heart valve, comprising: forming an outer skirt by attaching a non-tissue layer to a tissue layer; and mounting the outer skirt to an outer surface of an annular frame.

Example 39. The method of any example herein, particularly example 38, further comprising preparing the tissue layer to have a fibrous side and a smooth side, wherein forming the outer skirt comprises attaching the non-tissue layer to the smooth side of the tissue layer, wherein mounting the outer skirt causes the fibrous side of the tissue layer to face away from the frame and the non-tissue layer face towards the frame.

Example 40. A method comprising: delivering a prosthetic device in a radially compressed state to a target location; and radially expanding the prosthetic device to a radially expanded state, wherein the prosthetic device is a prosthetic valve according to any one of examples 1-10 and 35-37.

Example 41. A method comprising sterilizing the prosthetic valve of any one of examples 1-10 and 35-37.

The features described herein with regard to any example can be combined with other features described in any one or more of the other examples, unless otherwise stated. For example, any one or more of the features of one prosthetic valve can be combined with any one or more features of another prosthetic valve.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A prosthetic valve, comprising:
a radially expandable and compressible frame;
a leaflet assembly comprising a plurality of leaflets coupled to the frame; and
an outer skirt positioned around an outer surface of the frame,
wherein the outer skirt comprises a tissue layer and a non-tissue layer.

2. The prosthetic valve of claim 1, wherein the tissue layer comprises a fibrous side and a smooth side, wherein the non-tissue layer is coupled to the smooth side of the tissue layer, wherein the outer skirt is positioned so that the fibrous side of the tissue layer faces away from the frame and the non-tissue layer faces towards the frame.

3. The prosthetic valve of claim 2, wherein the non-tissue layer is attached to the smooth side of the tissue layer via an adhesive.

4. The prosthetic valve of claim 2, wherein the tissue layer comprises a plurality of slits through which blood can move from the fibrous side to the smooth side such as to promote tissue ingrowth within the plurality of slits.

5. The prosthetic valve of claim 4, wherein the plurality of slits are elongated in an axial direction.

6. The prosthetic valve of claim 1, wherein the non-tissue layer covers at least a majority of the tissue layer.

7. The prosthetic valve of claim 1, wherein the tissue layer comprises pericardial tissue.

8. The prosthetic valve of claim 1, wherein the non-tissue layer is fixedly attached to the tissue layer via one or more sutures.

9. The prosthetic valve of claim 1, wherein the tissue layer has a thickness between 50 μm and 100 μm, inclusive.

10. The prosthetic valve of claim 1, wherein the non-tissue layer comprises any one of polytetrafluoroethylene (PTFE), thermoplastic polyurethane (TPU), and silicone.

11. A method of making a prosthetic heart valve, comprising:
forming an outer skirt by attaching a non-tissue layer to a tissue layer; and
mounting the outer skirt to an outer surface of an annular frame.

12. The method of claim 11, further comprising preparing the tissue layer to have a fibrous side and a smooth side, wherein forming the outer skirt comprises attaching the non-tissue layer to the smooth side of the tissue layer, wherein mounting the outer skirt causes the fibrous side of the tissue layer to face away from the frame and the non-tissue layer face towards the frame.

13. The method of claim 12, wherein the act of forming the outer skirt comprises gluing the non-tissue layer to the smooth side of the tissue layer.

14. The method of claim 12, wherein the act of preparing the tissue layer comprises laser milling the tissue layer until the tissue layer has a thickness between 50 μm and 100 μm, inclusive.

15. The method of claim 14, wherein the act of laser milling comprises applying a femtosecond laser to the smooth side of the tissue layer.

16. The method of claim 14, wherein the act of laser milling comprises applying a thermal laser to the fibrous side of the tissue layer.

17. The method of claim 12, wherein the act of preparing the tissue layer comprises fraying the fibrous side of the tissue layer via chemical modification.

18. The method of claim 12 wherein the act of preparing the tissue layer comprises fraying the fibrous side of the tissue layer by mechanically altering the fibrous side.

19. The method of claim 12, wherein the act of preparing the tissue layer comprises forming a plurality of slits on the tissue layer through which blood can move from the fibrous side to the smooth side.

20. A method comprising:
delivering a prosthetic device in a radially compressed state to a target location; and
radially expanding the prosthetic device to a radially expanded state, wherein the prosthetic device comprises:
a radially expandable and compressible frame;
a leaflet assembly comprising a plurality of leaflets coupled to the frame; and
an outer skirt positioned around an outer surface of the frame, wherein the outer skirt comprises a tissue layer and a non-tissue layer.
